(19) United States
(12) Patent Application Publication
(10) Pub. No.: US 2026/0290076 A1
(43) Pub. Date: Sep. 24, 2026

(54) BLINK ESTIMATION DEVICE, LEARNING DEVICE, BLINK ESTIMATION METHOD, LEARNING METHOD, AND PROGRAM

(71) Applicant: NIPPON TELEGRAPH AND TELEPHONE CORPORATION, Tokyo (JP)

(72) Inventors: Ryota NISHIZONO, Musashino-shi, Tokyo (JP); Makio KASHINO, Musashino-shi, Tokyo (JP); Naoki SAIJO, Musashino-shi, Tokyo (JP)

(73) Assignee: NIPPON TELEGRAPH AND TELEPHONE CORPORATION, Tokyo (JP)

(21) Appl. No.: 18/998,036

(22) PCT Filed: Aug. 2, 2022
(86) PCT No.: PCT/JP2022/029617

§ 371 Date: Jan. 23, 2025

(51) Int. Cl. G06V 40/18; G06F 3/01

(52) U.S. Cl. CPC G06V 40/193; G06F 3/013

(57) ABSTRACT

A time during which an eyelid performs a movement having a physiological characteristic of a spontaneous blink is estimated by using image information indicating the movement of the eyelid, and information indicating the time is output.

### TECHNICAL FIELD

The present invention relates to a technique of detecting a blink.

### BACKGROUND ART

There is known a technique of detecting a time at which a blink has occurred from image information obtained by an eye camera (see, for example, Non Patent Literature 1).

### CITATION LIST

### Non Patent Literature

Non Patent Literature 1: Schweizer, Theresa, Thomas Wyss, and Rahel Gilgen-Ammann. “Eyeblink detection in the field: a proof of concept study of two mobile optical eye-trackers.” Military Medicine 187.3-4 (2022): e404-e409.

### SUMMARY OF INVENTION

### Technical Problem

However, it is difficult to accurately estimate a time at which a blink has occurred under various environments.

The present invention provides a technique of accurately estimating a time at which a blink has occurred even under various environments.

### Solution to Problem

A time during which an eyelid performs a movement having a physiological characteristic of a spontaneous blink is estimated by using image information indicating the movement of the eyelid, and information indicating the time is output.

### Advantageous Effects of Invention

This makes it possible to accurately estimate a time at which a blink has occurred even under various environments.

### BRIEF DESCRIPTION OF DRAWINGS

FIG. 1A is a block diagram showing a functional configuration of a learning device according to an embodiment. FIG. 1B is a block diagram showing a functional configuration of a blink estimation device according to an embodiment.

FIG. 2 is a block diagram showing a functional configuration of a blink estimation device according to an embodiment.

FIG. 3 is a block diagram showing a hardware configuration of a device according to an embodiment.

### DESCRIPTION OF EMBODIMENTS

Hereinafter, embodiments of the present invention will be described with reference to the drawings.

### First Embodiment

First, a first embodiment of the present invention will be described.

### <Configuration>

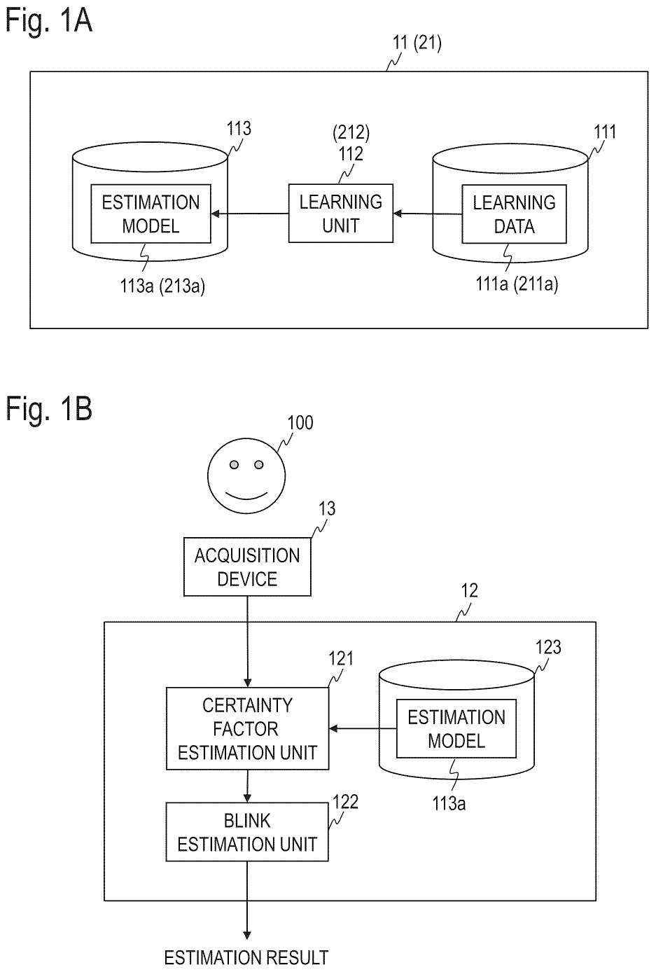

As shown in FIG. 1A, a learning device 11 of the present embodiment includes storage units 111 and 113 and a learning unit 112 and obtains an estimation model 113a by learning processing (learning processing of machine learning) using learning data 111a.

As shown in FIG. 1B, a blink estimation device 12 of the present embodiment includes a certainty factor estimation unit 121, a blink estimation unit 122, and a storage unit 123 and obtains and outputs an estimation result of a blink of a user 100 by using the estimation model 113a obtained by the learning device 11 and information indicating a movement of an eyelid of the user 100 obtained by an acquisition device 13.

The acquisition device 13 may be any device as long as the device acquires the information indicating the movement of the eyelid of the user 100. For example, the acquisition device 13 may be an eye camera, eye tracker, or image sensor that acquires image information (moving image information) indicating the movement of the eyelid of the user 100, may be a biometric sensor that acquires a biometric signal (e.g. a myoelectric potential signal) accompanied by the movement of the eyelid of the user 100, or may be a sensor that acquires a position, speed, acceleration, or the like of the eyelid of the user 100 (e.g. a position sensor, a speed sensor, or an acceleration sensor). The acquisition device 13 may also be a device that acquires information indicating a movement of the eyelids of both eyes of the user 100 or may be a device that acquires information indicating a movement of the eyelid of one eye of the user 100.

### <Learning Processing>

Next, the learning processing by the learning device 11 (FIG. 1A) of the present embodiment will be described.

The learning device 11 of the present embodiment learns the estimation model 113a for estimating a time during which a movement having a physiological characteristic of a spontaneous blink is performed from information indicating a movement of an eyelid.

A blink is a type of opening/closing movement of an eyelid of an eye of animals (including humans) and is roughly divided into a conscious blink and an unconscious blink. The conscious blink is called “voluntary blink”. Meanwhile, the unconscious blink is further divided into two types: “reflex blink” (a blink occurring when something comes near the eye, for example); and “spontaneous blink” (a blink occurring unconsciously and naturally). One of physiological characteristics of those blinks is a time length (length of duration) of the opening/closing movement, and the time length of the opening/closing movement at the time of blinking falls within a predetermined range in many cases. In general, the time length (length of duration) of the reflex blink is shorter than that of the spontaneous blink. The time length of the voluntary blink is substantially equal to or longer than that of the spontaneous blink. There is no upper limit on the time length of the voluntary blink. By using those characteristics, if the eyelid performs an opening/closing movement having a time length physiologically corresponding to a blink, it can be estimated that the opening/closing movement is a blink, and, if not, it can be estimated that the opening/closing movement is not a blink. Therefore, it is possible to accurately estimate a time at which a blink has occurred even under various environments. Here, a short one of the time lengths of the reflex blink, the spontaneous blink, and the voluntary blink falls within a range of 40 ms or more and less than 500 ms. Then, assuming that a shorter one of the reflex blink and the voluntary blink does not occur, a blink having a time length within the range of 40 ms or more and less than 500 ms can be extracted as the spontaneous blink. By performing processing so as to extract the spontaneous blink in this manner, it is possible to suppress error detection due to a problem of an image caused by ambient light or vibration and to accurately estimate the time at which a blink has occurred under various environments. The opening/closing movement of the eyelid is a series of movements in which the eye transitions from an eye-open state to an eye-closed state and transitions from the eye-closed state to the eye-open state again. The eye-closed is a state in which the eyelid is closed, and the eye-open is a state in which the eyelid is open. For example, the time length of the opening/closing movement is a time length from a time point when the eye-open state starts transitioning to the eye-closed state to a time point when the eye-closed state ends transitioning to the eye-open state.

Another physiological characteristic of a blink is that eyelids of both eyes perform an opening/closing movement. The characteristic that eyelids of both eyes perform an opening/closing movement may indicate, for example, that the eyelids of both the eyes perform the opening/closing movement simultaneously or substantially simultaneously or that both the eyes perform an opening/closing movement in which both the eyes are closed simultaneously or substantially simultaneously. By using the characteristic, if the eyelids of both the eyes perform the opening/closing movement, it can be estimated that the opening/closing movement is a blink, and, if not, it can be estimated that the opening/closing movement is not a blink. Therefore, it is possible to accurately estimate the time at which a blink has occurred even under various environments.

The estimation can also be made by combining the above two physiological characteristics. That is, if the eyelids of both the eyes perform an opening/closing movement whose time length physiologically corresponds to a spontaneous blink, it can be estimated that the opening/closing movement is a blink, and, if not, it can be estimated that the opening/closing movement is not a blink. By combining the above two physiological characteristics, it is possible to more accurately estimate the time at which a blink has occurred even under various environments.

The estimation model 113a of the present embodiment is a model that estimates a certainty factor indicating a degree of certainty of closing an eye or a degree of certainty of opening an eye on the basis of the information indicating the movement of the eyelid. As described later, such a certainty factor can be used to determine whether or not an opening/closing movement of the eyelid has the above physiological characteristics of a blink. Therefore, the estimation model 113a of the present embodiment can be considered as a model for estimating a time during which a movement having the above physiological characteristic of a blink is performed from the information indicating the movement of the eyelid. The certainty factor may be a discontinuous value indicated by binary values, may be a discontinuous value indicated by three or more values, or may be a continuous value. The certainty (probability) of closing the eye may be higher as the certainty factor is higher, the certainty of closing the eye may be higher as the certainty factor is lower, the certainty of opening the eye may be higher as the certainty factor is higher, or the certainty of opening the eye may be higher as the certainty factor is lower. The estimation model 113a may be any model as long as the model receives the information indicating the movement of the eyelid as an input and obtains and outputs the certainty factor. The estimation model 113a may be, for example, a model based on deep learning, a hidden Markov model, a support vector machine, or another known classifier. As an example, the estimation model 113a may be, for example, a deep convolutional neural network (DCNN) that obtains and outputs the certainty factor indicating the degree of certainty of closing the eye on the basis of information indicating each frame image of a moving image showing the movement of the eyelid. However, the present invention is not limited thereto.

The storage unit 111 stores the learning data 111a for obtaining the estimation model 113a by the learning processing. The learning data 111a depends on the estimation model 113a and includes at least learning information indicating the movement of the eyelid. The learning information indicating the movement of the eyelid is, for example, time-series information. For example, the learning information indicating the movement of the eyelid may be image information indicating the movement of the eyelid, may be a biometric signal accompanied by the movement of the eyelid, or may be the position, speed, acceleration, or the like of the eyelid. The learning data 111a may be supervised learning data or unsupervised learning data. For example, the estimation model 113a is a set of the learning information indicating the movement of the eyelid and a correct answer label corresponding thereto. For example, in a case where the estimation model 113a is the DCNN that obtains and outputs the certainty factor indicating the degree of certainty of closing the eye on the basis of information indicating each frame image of a moving image showing the movement of the eyelid, a set of each learning frame image of the moving image showing the movement of the eyelid (learning image information indicating the movement of the eyelid) and a correct answer label corresponding thereto is the learning data 111a. The correct answer label of the present embodiment may be a label indicating whether the eye is closed or open, may be a label indicating the certainty factor, or may be a label indicating a function value of the certainty factor. Note that whether the eye is closed or open and the certainty factor are defined based on a unified reference. For example, if a pupil is entirely hidden, it may be determined that the eye is closed, and, if not, it may be determined that the eye is open.

The learning unit 112 executes the learning processing using the learning data 111a stored in the storage unit 111, obtains the estimation model 113a, and stores the estimation model in the storage unit 113. The learning processing may be any processing. For example, the learning unit 112 may perform the learning processing by using only the learning data 111a stored in the storage unit 111 or may perform transfer learning using the learning data 111a stored in the storage unit 111 on the basis of a large-scale learned DCNN such as Resnet-50. In the transfer learning, for example, learning data, which is obtained by changing information included in the learning data 111a, adding noise to the information, removing a part of the information, or moving or rotating the information, is added as new learning data (so-called data augmentation), and the estimation model 113a is learned by using the original learning data 111a and the new learning data. For example, in a case where the learning data 111a is a set of learning image information indicating the movement of the eyelid (e.g. each frame image of the moving image showing the movement of the eyelid) and a correct answer label corresponding thereto, a set of new image information (e.g. a frame image) obtained by changing luminance or color of the image information (e.g. the frame image), adding noise to the image information, removing a part of the image information, or moving or rotating the image information, the original image information (e.g. the frame image), and correct answer labels corresponding thereto may be added as the new learning data.

The estimation model 113a obtained as described above is also stored in the storage unit 123 of the blink estimation device 12 (FIG. 1B).

### <Blink Estimation Processing>

Next, blink estimation processing by the blink estimation device 12 (FIG. 1B) of the present embodiment will be described.

The acquisition device 13 acquires information indicating a movement of the eyelid of the user 100. The information indicating the movement of the eyelid of the user 100 is, for example, time-series information. For example, the acquisition device 13 may acquire image information indicating the movement of the eyelid of the user 100, may acquire a biometric signal accompanied by the movement of the eyelid of the user 100, or may acquire the position, speed, acceleration, or the like of the eyelid of the user 100. Note that, the type of the information indicating the movement of the eyelid of the user 100 is the same as the type of the learning information indicating the movement of the eyelid included in the learning data 111a described above. For example, in a case where the estimation model 113a is the DCNN that obtains and outputs the certainty factor indicating the degree of certainty of closing the eye on the basis of information indicating each frame image of a moving image showing the movement of the eyelid (image information indicating the movement of the eyelid), the acquisition device 13 acquires each frame image of a moving image showing the movement of the eyelid of the user 100 (image information indicating the movement of the eyelid of the user 100). The acquisition device 13 may acquire information indicating a movement of the eyelids of both the eyes of the user 100 or may acquire information indicating a movement of the eyelid of one eye of the user 100. Note that, as described later, if the blink estimation unit 122 uses a physiological characteristic of a blink that the eyelids of both the eyes perform an opening/closing movement, the acquisition device 13 needs to acquire information indicating a movement of the eyelids of both the eyes of the user 100. The information indicating the movement of the eyelid of the user 100 acquired by the acquisition device 13 is input to the certainty factor estimation unit 121 (step S13).

The certainty factor estimation unit 121 applies the input information indicating the movement of the eyelid of the user 100 to the estimation model 113a extracted from the storage unit 123 and obtains and outputs a certainty factor indicating a degree of certainty that the eye of the user 100 is closed or a degree of certainty that the eye is open. For example, in a case where the information indicating the movement of the eyelid of the user 100, which is input to the certainty factor estimation unit 121, is time-series information, the certainty factor estimation unit 121 outputs time-series information of the certainty factor corresponding to the time-series information of the information indicating the movement of the eyelid of the user 100. In a case where the information indicating the movement of the eyelids of both the eyes of the user 100 is input to the certainty factor estimation unit 121, the certainty factor estimation unit 121 may obtain and output a certainty factor for each of the eyelids of both the eyes or may obtain and output a certainty factor for the eyelid of one of the eyes. However, as described later, if the blink estimation unit 122 uses a physiological characteristic of a blink that the eyelids of both the eyes perform an opening/closing movement, the certainty factor estimation unit 121 needs to obtain and output the certainty factor for each of the eyelids of both the eyes. In a case where the information indicating the movement of one eyelid of the user 100 is input to the certainty factor estimation unit 121, the certainty factor estimation unit 121 obtains and outputs a certainty factor for the eyelid of the one eye. The certainty factor output from the certainty factor estimation unit 121 is input to the blink estimation unit 122 (step S121).

The blink estimation unit 122 estimates a time during which the eyelid of the user 100 performs a movement having the physiological characteristic of a spontaneous blink (e.g. a time section in which the movement is performed or any time point belonging to the time section (e.g. a start time point, an end time point, a center time point, or a set of the start time point and the end time point)) on the basis of the input certainty factor and the above physiological characteristic of a blink and outputs information indicating the time as an estimation result (step S122). Specific examples of this processing will be described below. These are merely examples and do not limit the present invention.

### Specific Example 1

In a specific example 1, it is assumed that the acquisition device 13 acquires information indicating a movement of the eyelids of both the eyes of the user 100 and that the certainty factor estimation unit 121 outputs a certainty factor for each of the eyelids of both the eyes, and the blink estimation unit 122 estimates a time during which the eyelids of both the eyes perform an opening/closing movement whose time length physiologically corresponds to a spontaneous blink and outputs the time as an estimation result.

Certainty factors CR(0), . . . , and CR(T-1) of the right eye and certainty factors CL(0), . . . , and CL(T-1) of the left eye are input to the blink estimation unit 122.

Here, T is a positive integer, t=0, . . . , T-1 is an integer index indicating a time, and a larger t indicates a newer time. First, the blink estimation unit 122 uses an appropriate threshold to binarize the certainty factors CR(0), . . . , and CR(T-1) of the right eye, thereby obtaining binarized certainty factors CR′(0), . . . , and CR′(T-1) of the right eye, and to binarize the certainty factors CL(0), . . . , and CL(T-1) of the left eye, thereby obtaining binarized certainty factors CL′(0), . . . , and CL′(T-1) of the left eye. For example, the blink estimation unit 122 uses one threshold TH to set CR′(t)=1 where CR(t) >TH, set CR′(t)=0 where CR(t)≤TH, set CL′(t)=1 where CL(t)>TH, and set CL′(t)=0 where CL(t)≤TH. Alternatively, the blink estimation unit 122 may obtain the binarized certainty factors CR′(0), . . . , and CR′(T-1) and the binarized certainty factors CL′(0), . . . , and CL′(T-1) by threshold processing for preventing chattering using hysteresis. In this case, for example, the blink estimation unit 122 uses two thresholds, i. e., an upper limit value THU and a lower limit value THL(where THU>THL) to set CR′(0)=1 where CR(0)>THU and set CR′(0)=0 where CR(0)≤THU. Further, for t=1, . . . , T-1, the blink estimation unit 122 sets CR′(t)=1 where CR′(t-1)=1 and CR(t)>THL, sets CR′(t)=0 where CR′(t-1)=1 and CR(t)≤THL, sets CR′(t)=1 where CR′(t-1)=0 and CR(t)>THU, and sets CR′(t)=0 where CR′(t-1)=0 and CR(t)≤THU. Similarly, in this case, for example, the blink estimation unit 122 uses the two thresholds, i.e., the upper limit value THU and the lower limit value THL to set CL′(0)=1 where CL(0)>THU and set CL′(0)=0 where CL(0)≤THU.

Further, for t=1, . . . , T-1, the blink estimation unit 122sets CL′(t)=1 where CL′(t-1)=1 and CL(t)>THL, sets CL′(t)=0 where CL′(t-1)=1 and CL(t)≤THL, sets CL′(t)=1 where CL′(t-1)=0 and CL(t)>THU, and sets CL′(t)=0where CL′(t-1)=0 and CL(t)≤THU (step S1221-1).

Next, the blink estimation unit 122 extracts time sections IR(0), . . . , and IR(R-1) in which the opening/closing movement whose time length physiologically corresponds to a spontaneous blink is performed from the binarized certainty factors CR′(0), . . . , and CR′(T-1) of the right eye and extracts time sections IL(0), . . . , and IL(L-1) in which the opening/closing movement whose time length physiologically corresponds to a spontaneous blink is performed from the binarized certainty factors CL′(0), . . . , and CL′(T-1) of the left eye. The time length of the opening/closing movement is defined based on a predetermined reference. Note that R and L are positive integers equal to or less than T. For example, in a case where the binarized certainty factor 1 indicates that the eyes are open, and the binarized certainty factor 0 indicates that the eyes are closed, a time length of a section in which 0 continues (e.g. a section “00 . . . 000” in . . . 100 . . . 0001 . . . ) may be set as the time length of the opening/closing movement, or a total time length of a time of a section in which 0 continues and a time of sections of a predetermined number of 1 existing before and after the section (e.g. a section “11100 . . . 000111” in 1111100 . . . 000111111 . . . ) may be set as the time length of the opening/closing movement. The time length physiologically corresponding to a spontaneous blink is, for example, 40 ms or more and less than 500 ms (step S1222-1).

Next, the blink estimation unit 122 obtains times I(0), . . . , and I(K-1) in which both the eyes perform the opening/closing movement whose time length physiologically corresponds to a spontaneous blink by using the time sections IR(0), . . . , and IR(R-1) in which the right eye performs the opening/closing movement whose time length physiologically corresponds to a spontaneous blink and the time sections IL(0), . . . , and IL(L-1) in which the left eye performs the opening/closing movement whose time length physiologically corresponds to a spontaneous blink and outputs information indicating the times I(0), . . . , and I(K-1) as an estimation result (estimation result showing a time during which a blink is performed). Note that K is a positive integer equal to or less than T. The time I(k) (where k∈{0, . . . , K-1} ) may be a time point or a time section. For example, in a case where there is a time section IL(i) that matches or approximates a time section IR(r) (where r∈{0, . . . , R-1} and i∈{0, . . . , L-1}), the blink estimation unit 122 may set the time section IL(i) that matches or approximates the time section IR(r) as the time I(k) (where k∈{0, . . . , K-1} ), may set any time point belonging to the time section IL(i) (e.g. a start time point, end time point, center time point, and set of the start time point and the end time point of the time section IL(i)) as the time I(k), may set the time section IR(r) as the time I(k), or may set any time point belonging to the time section IR(r) as the time I(k). Alternatively, for example, the blink estimation unit 122 may set time sections in which the time sections IR(0), . . . and IR(R-1) and the time sections IL(0), . . . , and IL(L-1) overlap as the times I(0), . . . , and I(K-1) or may set any time point belonging to the overlapping time sections as the time I(k). That is, the blink estimation unit may set time sections belonging to the time sections IR(0), . . . , and IR(R-1) and belonging to the time sections IL(0), . . . , and IL(L-1) as the times I(0), . . . , and I(K-1) or may set any time point belonging to the time sections IR(0), . . . , and IR(R-1) and belonging to the time sections IL(0), . . . , and IL(L-1) as the time I(k). In a case where there is no time sections IR(0), . . . , and IR(R-1) or time sections IL(0), . . . , and IL(L-1), the blink estimation unit 122 may output an estimation result showing that there is no time during which a blink is performed. Alternatively, in a case where there is no time sections IR(0), . . . , and IR(R-1), the blink estimation unit 122 may set the time section IL(i) as the time I(k) or may set any time point belonging to the time section IL(i) as the time I(k). Similarly, in a case where there is no certainty factors CL(0), . . . ,and CL(T-1), the blink estimation unit 122 may set the time section IR(r) as the time section I(k) or may set any time point belonging to the time section IR(r) as the time I(k) (step S1223-1).

Note that the certainty factors CR(0), . . . , and CR(T-1) and certainty factors CL(0), . . . , and CL(T-1) having high reliability may not be obtained depending on an environment such as an illumination condition. In such a case, the blink estimation unit 122 may obtain the times I(0), . . . , and I(K-1) as described above by using only the certainty factors CR(0), . . . , and CR(T-1) and the certainty factors CL(0), . . . , and CL(T-1) whose reliability satisfies the reference. Alternatively, in a case where the reliability of the certainty factors CR(0), . . . , and CR(T-1) satisfies the reference, but the reliability of the certainty factors CL(0), . . . , and CL(T-1) does not satisfy the reference, the blink estimation unit 122 may set the time section IR(r) as the time I(k) or may set any time point belonging to the time section IR(r) as the time I(k). Similarly, in a case where the reliability of the certainty factors CL(0), . . . , and CL(T-1) satisfies the reference, but the reliability of the certainty factors CR(0), . . . , and CR(T-1) does not satisfy the reference, the blink estimation unit 122 may set the time section IL(i) as the time I(k) or may set any time point belonging to the time section IL(i) as the time I(k). Further, in a case where neither the reliability of the certainty factors CR(0), . . . , and CR(T-1) nor the reliability of the certainty factors CL(0), . . . , and CL(T-1) satisfies the reference, the blink estimation unit 122 may perform error processing, for example, may output no estimation result. Note that the reliability of the certainty factors may be any reliability. For example, if the size of the information indicating the movement of the eyelids of the corresponding user 100 (e.g. luminance of the image information) is below a saturation value, it may be determined that the reliability of the certainty factors satisfies the reference, and, if not, it may be determined that the reliability of the certainty factors does not satisfy the reference.

### Specific Example 2

In the specific example 1, the blink estimation unit 122 binarizes the certainty factors CR(0), . . . , and CR(T-1) of the right eye and the certainty factors CL(0), . . . , and CL(T-1) of the left eye (step S1222-1) and then extracts the times I(0), . . . , and I(K-1). However, the blink estimation unit 122 may obtain the times I(0), . . . , and I(K-1) without binarizing the certainty factors CR(0), . . . , and CR(T-1) of the right eye or the certainty factors CL(0), . . . , and CL(T-1) of the left eye.

In this case, the certainty factors CR(0), . . . , and CR(T-1) of the right eye and the certainty factors CL(0), . . . , and CL(T-1) of the left eye are input to the blink estimation unit 122. The blink estimation unit 122 extracts the time sections IR(0), . . . , and IR(R-1) in which the opening/closing movement whose time length physiologically corresponds to a spontaneous blink is performed from the certainty factors CR1(0), . . . , and CR(T-1) of the right eye and extracts the time sections IL(0), . . . , and IL(L-1) in which the opening/closing movement whose time length physiologically corresponds to a spontaneous blink is performed from the certainty factors CL(0), . . . , and CL(T-1) of the left eye. For example, the blink estimation unit 122 compares a predetermined threshold with the certainty factors CR(0), . . . , and CR(T-1), then detects the time sections IR(0), . . . , and IR(R-1) in which the opening/closing movement whose time length physiologically corresponds to a spontaneous blink is performed, and compares a predetermined threshold with the certainty factors CL(0), . . . , and CL(T-1), then detects the time sections IL(0), . . . , and IL(L-1) in which the opening/closing movement whose time length physiologically corresponds to a spontaneous blink is performed. For example, in a case where the eyes are open when the certainty factor is higher, whereas the eyes are closed when the certainty factor is lower, the blink estimation unit 122 may extract time sections in which the certainty factor CR(t) is continuously below the threshold and, among those time sections, set time sections whose time length physiologically corresponds to a spontaneous blink as the time sections IR(0), . . . , and IR(R-1). Similarly, in this case, the blink estimation unit 122 may extract, for example, time sections in which the certainty factor CL(t) is continuously below the threshold and, among those time sections, set time sections whose time length physiologically corresponds to a spontaneous blink as the time sections IL(0), . . . , and IL(L-1) (step S1222-2).

Thereafter, the blink estimation unit 122 executes step S1223-1 and outputs information indicating the times I(0), . . . , and I(K-1) as an estimation result. The other points are the same as those in the specific example 1.

### Specific Example 3

In a specific example 3, the blink estimation unit 122 estimates a time during which one eyelid performs an opening/closing movement whose time length physiologically corresponds to a spontaneous blink and outputs the time as an estimation result.

At least one of the certainty factors CR(0), . . . , and CR(T-1) of the right eye and the certainty factors CL(0), . . . , and CL(T-1) of the left eye is input to the blink estimation unit 122. The blink estimation unit 122 binarizes the certainty factors CR(0), . . . , and CR(T-1) of the right eye by using an appropriate threshold to obtain the binarized certainty factors CR′(0), . . . , and CR′(T-1) of the right eye, binarizes the certainty factors CL(0), . . . , and CL(T-1) of the left eye to obtain the binarized certainty factors CL′(0), . . . , and CL′(T-1) of the left eye, or obtains both the binarized certainty factors CR′(0), . . . , and CR′(T-1) and the binarized certainty factors CL′(0), . . . , and CL′(T-1) (step S1222-3).

The blink estimation unit 122 may extract, from the binarized certainty factors CR′(0), . . . , and CR′(T-1) of the right eye, the time section IR(r) in which the opening/closing movement whose time length physiologically corresponds to a spontaneous blink is performed as the time I(k) or may set any time point belonging to the time section IR(r) as the time I(k). Alternatively, the blink estimation unit 122 may extract, from the binarized certainty factors CL′(0), . . . , and CL′(T-1) of the left eye, the time section IL(i) in which the opening/closing movement whose time length physiologically corresponds to a spontaneous blink is performed as the time I(k) or may set any time point belonging to the time section IL(i) as the time I(k). Alternatively, the blink estimation unit 122 may obtain the times I(0), . . . , and I(K-1) as described above by using only the certainty factors CR(0), . . . , and CR(T-1) and certainty factors CL(0), . . . , and CL(T-1) whose reliability satisfies the reference. Alternatively, in a case where the reliability of the certainty factors CR(0), . . . , and CR(T-1) satisfies the reference, but the reliability of the certainty factors CL(0), . . . , and CL(T-1) does not satisfy the reference, the blink estimation unit 122 may obtain the times I(0), . . . , and I(K-1) as described above by using only the time sections IR(0), . . . , and IR(R-1). Similarly, in a case where the reliability of the certainty factors CL(0), . . . , and CL(T-1) satisfies the reference, but the reliability of the certainty factors CR(0), . . . , and CR(T-1) does not satisfy the reference, the blink estimation unit 122 may obtain the times I(0), . . . , and I(K-1) as described above by using only the time sections IL(0), . . . , and IL(L-1). The blink estimation unit 122 outputs information indicating the times I(0), . . . , and I(K-1) obtained in this manner as an estimation result. Further, in a case where neither the reliability of the certainty factors CR(0), . . . , and CR(T-1) nor the reliability of the certainty factors CL(0), . . . , and CL(T-1) satisfies the reference, the blink estimation unit 122 may perform error processing, for example, may output no estimation result (step S1223-3).

### Specific Example 4

In the specific example 3, the blink estimation unit 122 binarizes the certainty factors CR(0), . . . , and CR(T-1) of the right eye and/or the certainty factors CL(0), . . . , and CL(T-1) of the left eye (step S1222-3) and then extracts the times I(0), . . . , and I(K-1). However, the blink estimation unit 122 may obtain the times I(0), . . . , and I(K-1) without binarizing the certainty factors CR(0), . . . , and CR(T-1) of the right eye or the certainty factors CL(0), . . . , and CL(T-1) of the left eye.

In this case, the certainty factors CR(0), . . . , and CR(T-1) of the right eye and/or the certainty factors CL(0), . . . , and CL(T-1) of the left eye are input to the blink estimation unit 122. For example, the blink estimation unit 122 may compare a predetermined threshold with the certainty factors CR(0), . . . , and CR(T-1) and set the time section IR(r) in which the opening/closing movement whose time length physiologically corresponds to a spontaneous blink is performed as the time I(k) or set any time point belonging to the time section IR(r) as the time I(k). Alternatively, for example, the blink estimation unit 122 may compare a predetermined threshold with the certainty factors CL(0), . . . , and CL(T-1) and set the time section IL(i) in which the opening/closing movement whose time length physiologically corresponds to a spontaneous blink is performed as the time I(k) or set any time point belonging to the time section IL(i) as the time I(k) (see the specific example 2). The other points are the same as those in the specific example 3.

### Specific Example 5

In a specific example 5, it is assumed that the acquisition device 13 acquires information indicating the movement of the eyelids of both the eyes of the user 100 and that the certainty factor estimation unit 121 outputs the certainty factor for each of the eyelids of both the eyes, and the blink estimation unit 122 estimates a time during which the eyelids of both the eyes perform an opening/closing movement and outputs the time as an estimation result.

The certainty factors CR(0), . . . , and CR(T-1) of the right eye and the certainty factors CL(0), . . . , and CL(T-1) of the left eye are input to the blink estimation unit 122. First, the blink estimation unit 122 uses an appropriate threshold to binarize the certainty factors CR(0), . . . , and CR(T-1) of the right eye, thereby obtaining the binarized certainty factors CR′(0), . . . , and CR′(T-1) of the right eye, and to binarize the certainty factors CL(0), . . . , and CL(T-1) of the left eye, thereby obtaining the binarized certainty factors CL′(0), . . . , and CL′(T-1) of the left eye (step S1221-1).

Next, the blink estimation unit 122 extracts the time sections IR(0), . . . , and IR(R-1) in which the opening/closing movement is performed from the binarized certainty factors CR′(0), . . . , and CR′(T-1) of the right eye and extracts the time sections IL(0), . . . , and IL(L-1) in which the opening/closing movement is performed from the binarized certainty factors CL′(0), . . . , and CL′(T-1) of the left eye. A difference from the specific example 1 is that time sections in which an opening/closing movement is performed are extracted as IR(0), . . . , and IR(R-1) and IL(0), . . . , and IL(L-1), regardless of whether or not a time length thereof physiologically corresponds to a spontaneous blink (step S1222-5).

Next, the blink estimation unit 122 obtains the times I(0), . . . , and I(K-1) in which both the eyes perform the opening/closing movement by using the time sections IR(0), . . . , and IR(R-1) in which the right eye performs the opening/closing movement and the time sections IL(0), . . . , and IL(L-1) in which the left eye performs the opening/closing movement having a time length and outputs information indicating the times I(0), . . . , and I(K-1) as an estimation result (estimation result showing a time during which a blink is performed). A difference from the specific example 1 is that information indicating the times I(0), . . . , and I(K-1) during which both the eyes perform the opening/closing movement is output as an estimation result, regardless of whether or not a time length thereof physiologically corresponds to a spontaneous blink (step S1223-5).

### Specific Example 6

In the specific example 5, the blink estimation unit 122 binarizes the certainty factors CR(0), . . . , and CR(T-1) of the right eye and/or the certainty factors CL(0), . . . , and CL(T-1) of the left eye (step S1222-5) and then extracts the times I(0), . . . , and I(K-1). However, the blink estimation unit 122 may obtain the times I(0), . . . , and I(K-1) without binarizing the certainty factors CR(0), . . . , and CR(T-1) of the right eye or the certainty factors CL(0), . . . , and CL(T-1) of the left eye.

In this case, the certainty factors CR(0), . . . , and CR(T-1) of the right eye and the certainty factors CL(0), . . . , and CL(T-1) of the left eye are input to the blink estimation unit 122. The blink estimation unit 122 extracts the time sections IR(0), . . . , and IR(R-1) in which the opening/closing movement is performed from the certainty factors CR(0), . . . , and CR(T-1) of the right eye and extracts the time sections IL(0), . . . , and IL(L-1) in which the opening/closing movement is performed from the certainty factors CL(0), . . . , and CL(T-1) of the left eye. A difference from the specific example 2 is that time sections in which the opening/closing movement is performed are extracted as IR(0), . . . , and IR(R-1) and IL(0), . . . , and IL(L-1), regardless of whether or not a time length thereof physiologically corresponds to a spontaneous blink (step S1222-6).

Thereafter, the blink estimation unit 122 obtains the times I(0), . . . , and I(K-1) in which both the eyes perform the opening/closing movement by using the time sections IR(0), . . . , and IR(R-1) in which the right eye performs the opening/closing movement and the time sections IL(0), . . . , and IL(L-1) in which the left eye performs the opening/closing movement and outputs information indicating the times I(0), . . . , and I(K-1) as an estimation result (estimation result showing a time during which a blink is performed) (step S1223-6).

### Experimental Results

Next, experimental results of the present embodiment will be shown. In this experiment, the blink estimation unit 122 performed the processing of the specific example 1 by using 9576 frame images of an eye showing a movement of an eyelid for each of moving images A and B as the information indicating the movement of the eyelid of the learning data 111a, and, in step S1221-1, the blink estimation unit 122 binarized certainty factors by using one threshold TH.

In the moving image A, 764 blinks visually confirmed by a human were detected, whereas the blink estimation device 12 detected 760 blinks, and a median value of time shifts between blink start times visually confirmed by a human and blink start times detected by the blink estimation device 12 was 30.4 milliseconds. In the moving image B, 171 blinks visually confirmed by a human were detected, whereas the blink estimation device 12 detected 174 blinks, and a median value of time shifts between blink start times visually confirmed by a human and blink start times detected by the blink estimation device 12 was 25.6 milliseconds. Both the moving images were acquired under ambient light severe for image processing, for example, brightness is significantly different in both light and dark directions because of outside, and shadow of eyelashes was generated even in one image. In such a case, only some blinks could be detected by adjusting parameters in a detection method of classical image processing. Meanwhile, in the method of the present embodiment, such excellent results were obtained without adjusting the parameters.

### Features of Present Embodiment

In the present embodiment, a time during which an eyelid performs a movement having a physiological characteristic of a spontaneous blink is estimated by using image information indicating the movement of the eyelid, and information indicating the time is output. As described above, the physiological characteristic of a spontaneous blink is considered in the present embodiment, and thus it is possible to accurately estimate a time at which a blink has occurred even under various environments (e.g. various types of ambient light). It is further possible to assume various environments by using the estimation model 113a based on machine learning, thereby further improving estimation accuracy. Further, even in a case where the number of pieces of the learning data 111a is small, it is possible to improve the estimation accuracy in various environments by using transfer learning for learning the estimation model 113a.

### Second Embodiment

Next, a second embodiment of the present invention will be described. The estimation model 113a of the first embodiment is a model that estimates a certainty factor indicating a degree of certainty of closing an eye or a degree of certainty of opening an eye on the basis of information indicating a movement of an eyelid. However, instead of this, the estimation model may be a model that estimates a time during which an eyelid performs a movement having a physiological characteristic of a spontaneous blink on the basis of information indicating a movement of an eyelid. Differences from the first embodiment will be mainly described below, and the same reference signs will be used for the matters that have already been described to simplify the description.

### <Configuration>

As shown in FIG. 1A, a learning device 21 of the present embodiment includes the storage units 111 and 113 and a learning unit 212 and obtains an estimation model 213a by learning processing using learning data 211a.

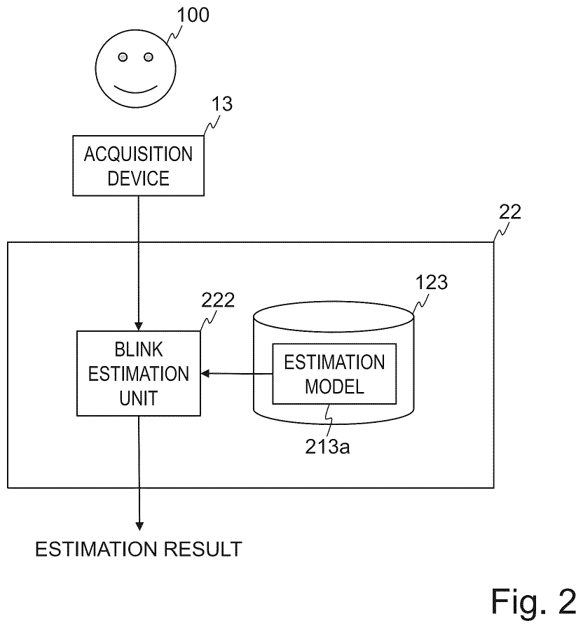

As shown in FIG. 2, a blink estimation device 22 of the present embodiment includes a blink estimation unit 222 and the storage unit 123, obtains and outputs an estimation result of a blink of the user 100 by using the estimation model 213a obtained by the learning device 21 and information indicating a movement of an eyelid of the user 100 obtained by the acquisition device 13.

### <Learning Processing>

Next, the learning processing by the learning device 21 (FIG. 1A) of the present embodiment will be described.

The learning device 21 of the present embodiment learns the estimation model 213a for estimating a time during which a movement having a physiological characteristic of a spontaneous blink is performed from information indicating a movement of the eyelid.

The estimation model 213a of the present embodiment is a model that estimates a time during which the eyelid performs a movement having a physiological characteristic of a spontaneous blink on the basis of the information indicating the movement of the eyelid. Specific examples of the physiological characteristic of a spontaneous blink are as described in the first embodiment. The estimation model 213a may be any model as long as the model receives the information indicating the movement of the eyelid as an input and obtains and outputs a time during which the eyelid performs a movement having a physiological characteristic of a spontaneous blink. The estimation model 213a may be, for example, a model based on deep learning, a hidden Markov model, a support vector machine, or another known classifier.

The storage unit 111 stores the learning data 211a for obtaining the estimation model 213a by the learning processing. The learning data 211a depends on the estimation model 213a and includes at least learning information indicating the movement of the eyelid. A specific example of the information indicating the movement of the eyelid is as described in the first embodiment. The learning data 211a may be supervised learning data or unsupervised learning data. For example, the estimation model 213a is a set of the learning information indicating the movement of the eyelid and a correct answer label corresponding thereto. The correct answer label of the present embodiment may be a label indicating whether or not the time is a time during which the eyelid performs a movement having a physiological characteristic of a spontaneous blink, may be a label indicating a probability that the time is a time during which the eyelid performs the movement having the physiological characteristic, or may be a label indicating a function value of the probability. Such a correct answer label can be generated, for example, by using an estimation result obtained by inputting the learning information indicating the movement of the eyelid to the blink estimation device 12 of the first embodiment.

The learning unit 212 executes the learning processing using the learning data 211a stored in the storage unit 111, obtains the estimation model 213a, and stores the estimation model in the storage unit 113. As described in the first embodiment, this learning processing may be any processing.

The estimation model 213a obtained as described above is also stored in the storage unit 123 of the blink estimation device 22 (FIG. 2).

### <Blink Estimation Processing>

Next, blink estimation processing by the blink estimation device 22 (FIG. 2) of the present embodiment will be described.

The acquisition device 13 acquires information indicating a movement of the eyelid of the user 100. The information indicating the movement of the eyelid of the user 100 acquired by the acquisition device 13 is input to the blink estimation unit 222 (step S23).

The blink estimation unit 222 applies the input information indicating the movement of the eyelid of the user 100 to the estimation model 213a extracted from the storage unit 123, estimates a time during which the eyelid of the user 100 performs a movement having a physiological characteristic of a spontaneous blink, and outputs information indicating the time as an estimation result (step S222).

### Features of Present Embodiment

In the present embodiment, a time during which an eyelid performs a movement having a physiological characteristic of a spontaneous blink is estimated by using image information indicating the movement of the eyelid, and information indicating the time is output. As described above, the physiological characteristic of a spontaneous blink is considered in the present embodiment, and thus it is possible to accurately estimate a time at which a blink has occurred even under various environments (e.g. various types of ambient light). In particular, the present embodiment uses the estimation model 213a that estimates a time during which the eyelid performs a movement having a physiological characteristic of a spontaneous blink on the basis of information indicating the movement of the eyelid. This makes it possible to use an output from the estimation model 213a as a blink estimation result as it is, thereby simplifying the blink estimation processing. The blink estimation processing can be managed only by the estimation model 213a stored in the blink estimation device 22 without considering a threshold, and thus it is easy to update the blink estimation processing. Further, it is possible to assume various environments (e.g. various types of ambient light) by using the estimation model 213a based on machine learning, thereby further improving estimation accuracy. Further, even in a case where the number of pieces of the learning data 211a is small, it is possible to improve the estimation accuracy in various environments by using transfer learning for learning the estimation model 213a.

### <Hardware Configuration>

The learning devices 11 and 21 and the blink estimation devices 12 and 22 in the embodiments are devices configured by a general-purpose or dedicated computer executing a predetermined program, the computer including a processor (hardware processor) such as a central processing unit (CPU) and a memory such as a random access memory (RAM) and a read only memory (ROM), for example. That is, the learning devices 11 and 21 and the blink estimation devices 12 and 22 in the embodiments include processing circuitry configured to implement each unit included therein, for example. This computer may include one processor and one memory or may include a plurality of processors and a plurality of memories. The program may be installed into the computer or may be recorded in the ROM or the like in advance. Further, some or all processing units may be configured by using electronic circuitry that independently implements a processing function, instead of electronic circuitry that implements a functional configuration by reading a program, such as a CPU.

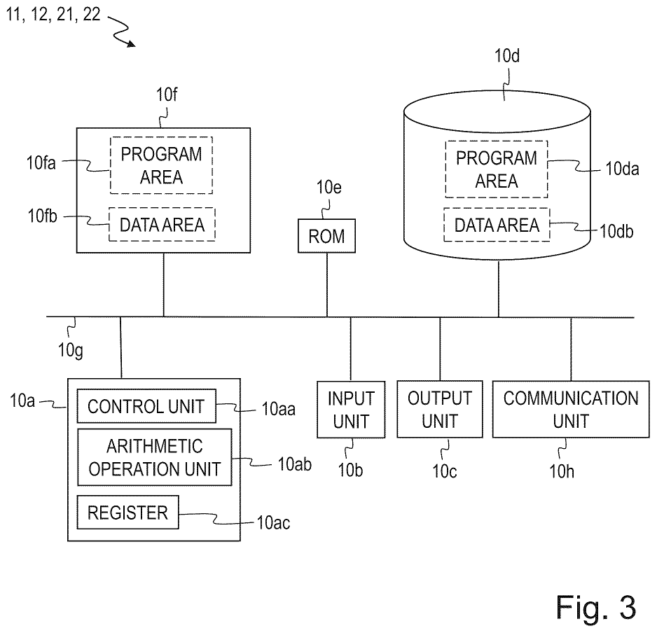

Electronic circuitry forming one device may include a plurality of CPUs.
FIG. 3 is a block diagram showing a hardware configuration of the learning devices 11 and 21 and the blink estimation devices 12 and 22 in the embodiments. As shown in FIG. 3, the learning devices 11 and 21 and the blink estimation devices 12 and 22 in this example include a central processing unit (CPU) 10a, an input unit 10b, an output unit 10c, a random access memory (RAM) 10d, a read only memory (ROM) 10e, an auxiliary storage device 10f, a communication unit 10h, and a bus 10g. The CPU 10a of this example includes a control unit 10aa, an arithmetic operation unit 10ab, and a register 10ac and performs various arithmetic operations in accordance with various programs read into the register 10ac. The input unit 10b is an input terminal, a keyboard, a mouse, a touch panel, or the like to which data is input. The output unit 10c is an output terminal, a display, or the like from which data is output. The communication unit 10h is a LAN card or the like controlled by the CPU 10a that has read a predetermined program. The RAM 10d is a static random access memory (SRAM), a dynamic random access memory (DRAM), or the like and has a program area 10da in which a predetermined program is stored and a data area 10db in which various kinds of data are stored. The auxiliary storage device 10f is, for example, a hard disk, a magneto-optical disc (MO), or a semiconductor memory and has a program area 10fa in which a predetermined program is stored and a data area 10fb in which various kinds of data are stored. The bus 10g connects the CPU 10a, the input unit 10b, the output unit 10c, the RAM 10d, the ROM 10e, the communication unit 10h, and the auxiliary storage device 10f such that information can be exchanged therebetween. The CPU 10a writes, into the program area 10da of the RAM 10d, the program stored in the program area 10fa of the auxiliary storage device 10f in accordance with a read operating system (OS) program. Similarly, the CPU 10a writes, into the data area 10db of the RAM 10d, the various kinds of data stored in the data area 10fb of the auxiliary storage device 10f. Addresses on the RAM 10d in which the program and the data have been written are stored in the register 10ac of the CPU 10a. The control unit 10aa of the CPU 10a sequentially reads those addresses stored in the register 10ac, reads the program and the data from the areas in the RAM 10d indicated by the read addresses, causes the arithmetic operation unit 10ab to sequentially execute arithmetic operations indicated by the program, and stores results of the arithmetic operations in the register 10ac. Such a configuration achieves functional configurations of the learning devices 11 and 21 and the blink estimation devices 12 and 22.

The above program can be recorded in a computer-readable recording medium. Examples of the computer-readable recording medium include a non-transitory recording medium. Examples of such a recording medium include a magnetic recording device, an optical disk, a magneto-optical recording medium, and a semiconductor memory.

The program is distributed by, for example, selling, transferring, or renting a portable recording medium such as a DVD or a CD-ROM in which the program is recorded. Further, the program may be stored in a storage device of a server computer, and the program may be distributed by transferring the program from the server computer to another computer via a network. As described above, the computer executing such a program first temporarily stores the program recorded in the portable recording medium or the program transferred from the server computer in a storage device of the computer, for example. Then, at the time of executing processing, the computer reads the program stored in the storage device thereof and executes processing according to the read program. In another mode of the program, the computer may read a program directly from the portable recording medium and execute processing according to the program, or alternatively, every time a program is transferred from the server computer to the computer, the computer may sequentially execute processing according to the received program. The above-described processing may be executed by a so-called application service provider (ASP) type service that implements a processing function only by an execution instruction and result acquisition without transferring the program from the server computer to the computer. Note that the program in the present embodiment includes information used for processing by an electronic computer and equivalent to the program (data or the like that is not a direct command to the computer but has a property that defines processing of the computer).

Although the present device is configured by executing a predetermined program in the computer in each embodiment, at least part of processing content may be implemented by hardware.

### Other Modification Examples

Note that the present invention is not limited to the above embodiments. For example, the user 100 may be an animal other than a human. The various types of processing described above may be performed not only in time series according to the description but also in parallel or individually according to the throughput of the device that performs the processing or as necessary. Further, it goes without saying that changes can be made as appropriate without departing from the contents described in the claims.

### REFERENCE SIGNS LIST

11, 21 Learning device
12, 22 Blink estimation device

1. A blink estimation device comprising processing circuitry configured to estimate a time during which an eyelid performs a movement having a physiological characteristic of a spontaneous blink by using information indicating the movement of the eyelid and outputs information indicating the time.

2. The blink estimation device according to claim 1, wherein
the blink estimation device estimates the time during which the eyelid performs an opening/closing movement whose time length physiologically corresponds to the spontaneous blink.

3. The blink estimation device according to claim 1, wherein:
the information indicating the movement of the eyelid is information indicating a movement of eyelids of both eyes; and
the processing circuitry estimates the time during which the eyelids of both the eyes perform an opening/closing movement.

4. The blink estimation device according to claim 1, wherein:
the information indicating the movement of the eyelid is information indicating a movement of eyelids of both eyes; and
the processing circuitry estimation unit estimates the time during which the eyelids of both the eyes perform an opening/closing movement whose time length physiologically corresponds to the spontaneous blink.

5. A learning device comprising processing circuitry configured to learn and output
an estimation model for estimating a time during which an eyelid performs a movement having a physiological characteristic of a spontaneous blink from information indicating the movement of the eyelid.

6. A blink estimation method by a blink estimation device, the blink estimation method comprising
estimating a time during which an eyelid performs a movement having a physiological characteristic of a spontaneous blink by using information indicating the movement of the eyelid and outputting information indicating the time.

7. A learning method by a learning device, the learning method comprising learning and outputting
an estimation model for estimating a time during which an eyelid performs a movement having a physiological characteristic of a spontaneous blink from information indicating the movement of the eyelid.

8. A non-transitory computer-readable recording medium storing a program for causing a computer to function as the blink estimation device according to claim 1.

9. A non-transitory computer-readable recording medium storing a program for causing a computer to function as the learning device according to claim 5.
